(19) United States
(12) Patent Application Publication
(10) Pub. No.: US 2022/0088379 A1
(43) Pub. Date: Mar. 24, 2022

(54) CARDIAC PACING VIA THE DISTAL PURKINJE SYSTEM WITH ULTRA-SHORT PULSE WIDTHS

(71) Applicant: Morton M. MOWER, Denver, CO (US)

(72) Inventor: Morton M. MOWER, Denver, CO (US)

(21) Appl. No.: 17/475,908

(22) Filed: Sep. 15, 2021

Related U.S. Application Data
(60) Provisional application No. 63/082,078, filed on Sep. 23, 2020.

(51) Int. Cl. A61N 1/365; A61N 1/05; A61N 1/378

(52) U.S. Cl. CPC A61N 1/365; A61N 1/378; A61N 1/056

(57) ABSTRACT

Methods for cardiac pacing in a human heart using a biphasic waveform having a first pulse having an anodal (positive) polarity followed by a second pulse having a cathodal (negative) polarity. Electrodes in the right bundle branch are used to stimulate the Purkinje fibers with low voltage, ultra-short short pulse widths using a fraction of the energy needed for capture enabling much longer battery life. Alternatively, biphasic anodal/cathodal waveforms are used to stimulate HIS bundle pacing of the mid-septum right bundle branch to enable retrograde conduction back through the atrioventricular (AV) node and down the left bundle thus enabling cardiac resynchronization from the right ventricle. The pacing stimulation applying a biphasic waveform with anodal-first component speeds conduction of pacing stimuli through the conduction system. A sinus node electrode may provide a defibrillation stimulus before the biphasic anodal/cathodal waveforms are applied in HIS bundle pacing.

### CROSS-REFERENCE TO RELATED APPLICATIONS

The present application claims priority of U.S. Application No. 63/082,078, filed Sep. 23, 2020, the entire contents of which is hereby incorporated by reference.

### BACKGROUND

### Field

Methods for cardiac pacing via the distal Purkinje system with ultra-short pulse widths and for cardiac pacing of the mid-septum right bundle branch to enable retrograde conduction back through the AV node and down the left bundle to enable cardiac resynchronization from the right ventricle.

### Description of the Related Art

Cardiac rhythm management (CRM) has used the same pacing waveforms for decades. A cathodal based pulse has been used to stimulate the left ventricle with known side effects. Research has shown that cathodal pacing causes inflammation and reduces cardiac contractility over time. Knowledge of the cardiac conduction system has expanded rapidly and revealed there is a need for improvement of these pacing waveforms.

Atrial fibrillation (AF) may cause sudden cardiac arrest and has been treated with pacemakers. Pacemakers provide pacing waveforms to electrodes implanted in the heart. A pacemaker may be a control box worn on a patient's body, with leads to the electrodes, may be surgically implanted under the skin, or may be a fully implantable device operated by battery power.

Traditional right ventricular (RV) pacing for the management of bracyarrhythmias has been pursued successfully for decades, although there remains debate regarding optimal pacing sites with respect to both hemodynamic and clinical outcomes. The deleterious effects of long-term RV apical pacing have been well recognized. This has generated interest in approaches providing more physiological stimulation, by HIS bundle pacing (HBP). Recent studies have also demonstrated the potential of HBP in patients with underlying left bundle branch block and cardiomyopathy. HBP holds promise as an attractive mode to achieve physiological pacing as HBP engages electrical activation of both ventricles and may avoid marked dysynchrony.

Accordingly, it is one object of the present disclosure to provide methods and systems for cardiac pacing in the HIS-Purkinje network using biphasic anodal/cathodal waveforms.

### SUMMARY

In an exemplary embodiment of the present disclosure, a method of cardiac pacing of a human heart is described, comprising: stimulating Purkinje fibers of a human heart by a biphasic waveform including an anodal pulse followed by a cathodal pulse.

In another exemplary embodiment, a method of cardiac pacing of a human heart is described, comprising: stimulating the HIS bundle of a human heart by a biphasic waveform including an anodal pulse followed by a cathodal pulse.

In a further exemplary embodiment, a method extending the lifetime of a pacemaker battery is described, comprising: generating, by a pulse generator of the pacemaker, a series of low voltage square wave biphasic waveforms each including an anodal pulse followed by a cathodal pulse, the biphasic waveforms having ultra-short pulse widths; applying the anodal pulse to an anodal electrode located in a human heart, wherein the anodal pulse has a positive amplitude V₁; applying the cathodal pulse to an cathodal electrode located in a human heart, wherein the cathodal pulse has a negative amplitude V₂, wherein V₁ and V₂ are lower in amplitude than an amplitude of a voltage V₃ applied to the cathodic electrode by a single phase pacing waveform; and stimulating the human heart by the series of biphasic waveforms.

In another exemplary embodiment, a method for cardiac resynchronization in a human heart is described, comprising: installing a large surface area electrode at a sinus node of the human heart; installing an anodal biventricular tip electrode at an atrioventricular node of the human heart; installing a cathodal biventricular ring electrode around a HIS bundle of the human heart; sensing, by a pacemaker connected to the anodal and cathodal electrodes, electrical signals which indicate an onset of atrial fibrillation; applying a positive pulse of voltage amplitude V₁ to the large surface area electrode; applying a pulse of positive amplitude V₂ to the anodal electrode followed by a negative pulse of amplitude V₃ to the cathodal electrode, wherein the amplitude of V₁ is greater than the amplitudes of V₂ and V₃, and wherein the amplitude of V₂ is less than or equal to the amplitude of V₃.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A illustrates a side view of a human heart.

FIG. 1B illustrates a cross-sectional view of the human heart.

FIG. 2A depicts a biphasic anodal/cathodal waveform.

FIG. 2B depicts a cathodal only waveform.

FIG. 2C depicts a biphasic cathodal/anodal waveform.

FIG. 3A is a threshold assessment chart for patients with permanent HIS bundle (HB) pacing showing a distinct pattern of capture for a wide zone of selective pacing that broadens with a shorter pulse width.

FIG. 3B is a threshold assessment chart for patients with permanent HIS bundle (HB) pacing showing a distinct pattern of obligatory selective capture.

FIG. 3C is a threshold assessment chart for patients with permanent HIS bundle (HB) pacing showing a distinct pattern of obligatory nonselective HB capture.

FIG. 4 is a graph comparing threshold voltage to pulse duration for HIS bundle pacing and right ventricle pacing.

FIG. 5 illustrates pulse duration to width of the HIS bundle zone for selective HB pacing subgroup in a box plot with median and interquartile range from 25% to 75%.

FIG. 6A illustrates pacing in the right and left bundle branch fibers within the HIS bundle at 2V resulting in nonselective HIS bundle pacing.

FIG. 6B illustrates pacing in the right and left bundle branch fibers within the HIS bundle at 1.5V resulting in selective HIS bundle pacing.

FIG. 6C illustrates pacing in the right and left bundle branch fibers within the HIS bundle at 1.0 volts capturing only the right bundle branch.

FIG. 7A shows a biventricular electrode having an extended bipole.

FIG. 7B shows a biventricular electrode having a split bipole.

FIG. 8A circuit diagram for biphasic pacing using a biventricular electrode for HIS bundle pacing.

FIG. 8B shows the amplitude with respect to time for a cathodal/anodal biphasic pulse and the evoked response.

FIG. 8C an electrocardiogram depicting the anodal and cathodal pulse waveforms.

FIG. 9A shows an external waveform stimulator.

FIG. 9B illustrates an implantable waveform stimulator showing the leads and the internal pacemaker circuit.

FIG. 9C shows a pacemaker circuit board.

FIG. 9D shows an oscilloscope picture of a biphasic anodal/cathodal pulse of the pacemaker of FIG. 9C.

FIG. 10 shows a graph of left ventricle pressure with respect to time.

FIG. 11 shows the change in speed versus pulse width at increasing voltage levels.

FIG. 12A shows the change in contractility in isolated muscle strips of a rabbit ventricle due to cathodal (left) and biphasic (right) pulses.

FIG. 12B shows the change in power in isolated muscle strips of a human atrium due to cathodal (left) and biphasic pulses (right).

FIG. 13A is a graph showing the change in conduction velocity (V/sec) with respect to stimulation pulse strengths of 2, 3 and 4 volts in an animal heart.

FIG. 13B is a graph of cardiac pressure with respect to cardiac volume for biphasic anodal/cathodal and cathodal only waveforms in a rabbit heart.

FIG. 14 shows comparison of cardiac pacing in pig hearts with a cathodal pulse only to pacing with a biphasic cathodal/anodal waveform.

FIG. 15A is a box and whiskers graph comparing the mean and standard deviation of the steady state preload-adjusted PWRmax.

FIG. 15B depicts pressure and volume loops in comparative stroke experiments using cathodal and biphasic pulses.

FIG. 16A shows a comparison of cathodal, biphasic and unpaced left ventricle diastolic volumes.

FIG. 16B shows the changes in percent fractional shortening at nine weeks for the comparison of FIG. 16A.

FIG. 17 shows an experiment on humans of using biphasic anodal/cathodal cardiac pacing in the atrium and in the ventricle for narrowing the paced QRS duration.

FIG. 18 shows a graph of mortality rates versus months from admission for patients having dual chamber sensing and stimulating greater than 40% of the time.

FIG. 19A shows that greater than 50% pacing increased the congestive heart failure risk.

FIG. 19B shows greater than 50% pacing increased the atrial fibrillation risk.

### DETAILED DESCRIPTION

Referring now to the drawings, wherein like reference numerals designate identical or corresponding parts throughout the several views, the artificial pacemaker is a medical device that is surgically implanted, most commonly in the subcutaneous tissues overlying the prepectoral fasci. About 98% of pacemakers are implanted due to a patient's inability to maintain an adequate heart rate due to a block somewhere within the intrinsic electrical conducting system (sinoatrial node, atrioventricular junction, His-Purkinje system).

The pacemaker system is composed of a pulse generator and one or more leads that connect the generator to the heart. Pacemaker batteries are designed to have predictable depletion over time, which can be monitored by their cell voltage and cell impedance. The life span of a particular generator in a particular patient is largely dependent on the percent of pacing, the programmed voltage, the pulse width, and electrical pacing impedances. Typically, batteries last 5 to 10 years. The leads are thin, flexible, insulated wires that conduct electrical impulses from the pacemaker generator to the heart and also relay electrical signals from the heart back to the generator. The majority of pacemaker leads are inserted transvenously from the generator to the endocardium (“transvenous leads”). Less commonly, leads are attached directly to the epicardial surface of the heart with either a helical screw or a suture-on-plaque electrode during heart surgery.

Fully implantable leadless pacemakers are also known, which are inserted directly into the right ventricle of the heart through a catheter inserted in the femoral artery. The implantable, leadless pacemaker is tethered to the endocardium of the right ventricle.

The two basic functions of the pacemaker system are pacing and sensing. Pacing refers to depolarization of the atria or ventricles, resulting from an impulse (typically 0.5 msec and 2 to 5 volts) delivered from the generator down a lead to the heart. Sensing refers to detection by the generator of intrinsic atrial or ventricular depolarization signals that are conducted up a lead. Sensed events are used by the pacemaker logic to appropriately time the pacing impulses.

Aspects of the present disclosure describe (1) cardiac pacing via the distal Purkinje system with a biphasic anodal/cathodal waveform having ultra-short pulse widths using a fraction of the energy needed for capture enabling much longer battery life, (2) HIS bundle pacing of the mid-septum right bundle branch with a biphasic anodal/cathodal waveform to enable retrograde conduction back through the atrioventricular (AV) node and down the left bundle thus enabling cardiac resynchronization from the right ventricle and (3) a method of extending the lifetime of a pacemaker battery by using a biphasic anodal/cathodal waveform to stimulate pacing, and a method for cardiac resynchronization in a human heart by applying a stimulation to a sinus node electrode followed by applying a biphasic anodal/cathodal pacing waveform to electrodes located in the atrioventricular node and around a HIS bundle.

The HIS bundle is a collection of heart muscle cells specialized for electrical conduction. As part of the electrical conduction system of the heart, it transmits the electrical impulses from the AV node (located between the atria and the ventricles) to the point of the apex of the fascicular branches via the bundle branches. The fascicular branches then lead to the Purkinje fibers, which provide electrical conduction to the ventricles, causing the cardiac muscle of the ventricles to contract at a paced interval.

Experimentation in non-human animals of numerous species indicate that monophasic anodal and biphasic anodal/cathodal pacing stimuli increases speed of conduction (with 0.5 msec/0.5 msec in the rabbit heart from 12% to 34% depending on the direction of the depolarization and the distance from the stimulating electrode), and increases the contractility of the myocardium (with the change in pressure per unit time (dP/dt) increasing by 32%). Also, the membrane potential, ATP and other products of cellular metabolism were increased, and insulin release in cell cultures was controlled in a manner different from the usual glucose-dependent mechanism.

In non-human animal studies, the rise in threshold associated with anodal pacing was theorized to be avoided by adding a cathodal pulse to the end of the anodal one, thereby producing a biphasic anodal/cathodal pulse. Increasing the speed of myocardial conduction and contractility through the use of a biphasic anodal/cathodal pulse was predicted to be beneficial for antitachycardia burst pacing for termination of atrial arrhythmias including fibrillation for enhancing cardiac output in heart failure from a right ventricular pacing site alone, and for improving the performance of pacemakers and defibrillators for conventional indications.

Experiments in rabbit hearts compared anodal, cathodal, and equiphasic biphasic pacing pulses. Anodal [A] stimulation pulses improved the electrical conduction at all the stimulus amplitudes tested in both longitudinal (e.g., 5 V 2-msec pulse: [A] 54.9+/−0.7 cm/sec; cathodal [C] 49.7+/−1.5 cm/sec) and transverse (e.g., 5 V 2 msec pulse: [A] 31.3+/−1.7 cm/sec; [C] 23.3+/−2.9 cm/sec) directions. Microelectrode recordings verified that increased conduction velocities of the anodal pulses were associated with faster upstrokes of the action potentials. The increased threshold associated with anodal pulses was overcome by using a biphasic (B) waveform, in effect adding a second phase (e.g., 2-msec pulse: [A]2.03+/−1.3 V; [C] 3.85+/−1.5 V; [B] 2.15+/−0.9 V). The conduction speeds achieved by the biphasic pulses were found to be comparable to the equivalent anodal pulses (e.g., 5 V 2-msec pulse: [B] 55.2+/−1.7 cm/sec longitudinal and 32.4+/−2.1 cm/sec transverse). This research demonstrated, that in non-human animal models, anodal and biphasic stimulation increased the conduction velocity and increased cardiac contractility.

Experiments in cardiac pacing in sheep were conducted in which myocardial infarction was induced in sheep by high coronary artery ligation. The animals exhibited increased left ventricular volume and reduced percent fractional shortening. Two weeks after the infarction, sheep were implanted with atrial-triggered, right ventricular pacemaker systems capable of pacing with cathodal (cathodal pulse) and biphasic (anodal pulse followed by cathodal pulse) waveforms, and randomly assigned to an initial mode. At three month intervals, the pacing system was switched to the alternative mode. Cardiac function was assessed at two to three week intervals through the use of echocardiograms. Successful pacing was confirmed over an average of eight weeks in each mode. Cathodal pulsing had neither beneficial nor deleterious effect on the diminished cardiac performance induced by myocardial infarction. When compared to the cathodal mode, biphasic pulsing improved cardiac performance as reflected by decrease of diastolic and systolic ventricular volumes, reduction in left ventricular systolic diameter, and increases in percent fractional shortening. When compared to the unpaced state after the myocardial infarction, the percent fractional shortening was significantly increased by biphasic pacing. Concordant trends in improvement in the other cardiac parameters were also observed for the biphasic mode. No ventricular tachyarrhythmias or mortality was associated with biphasic stimulation. Biphasic pulsing elicited significant benefits in cardiac performance.

The effect of the biphasic anodal/cathodal pacing stimuli are theorized to be mediated by hyper-polarization of the cells prior to their actual depolarization. This is because this first anodal phase is non-stimulatory, but preconditions the tissue by increasing the membrane potential, so that when stimulation does occur on the “break” of the anodal coincident with the “make” of the cathodal phase, the depolarization occurs from a more electronegative point, the phase zero is steeper, more sodium rushes in, the depolarization is stronger, conduction speed is increased, more calcium is exchanged for the sodium and contractility is enhanced in addition to other intra-cellular effects.

The heart is regulated by both neural and endocrine control, yet it is capable of initiating its own action potential followed by muscular contraction. The conductive cells within the heart establish the heart rate and transmit it through the myocardium. The contractile cells contract and propel the blood. The normal path of transmission for the conductive cells is the sinoatrial (SA) node, internodal pathways, atrioventricular (AV) node, atrioventricular (AV) bundle of HIS, bundle branches, and Purkinje fibers.

The Purkinje fibers are located in the inner ventricular walls of the heart, just beneath the endocardium in a space called the sub endocardium. The Purkinje fibers are specialized conducting fibers composed of electrically excitable cells that are larger than cardiomyocytes with fewer myofibrils and many mitochondria and which conduct cardiac action potentials more quickly and efficiently than any other cells in the heart. These Purkinje fibers allow the conduction system of the heart to create synchronized contractions of its ventricles, and are, therefore, essential for maintaining a consistent heart rhythm.

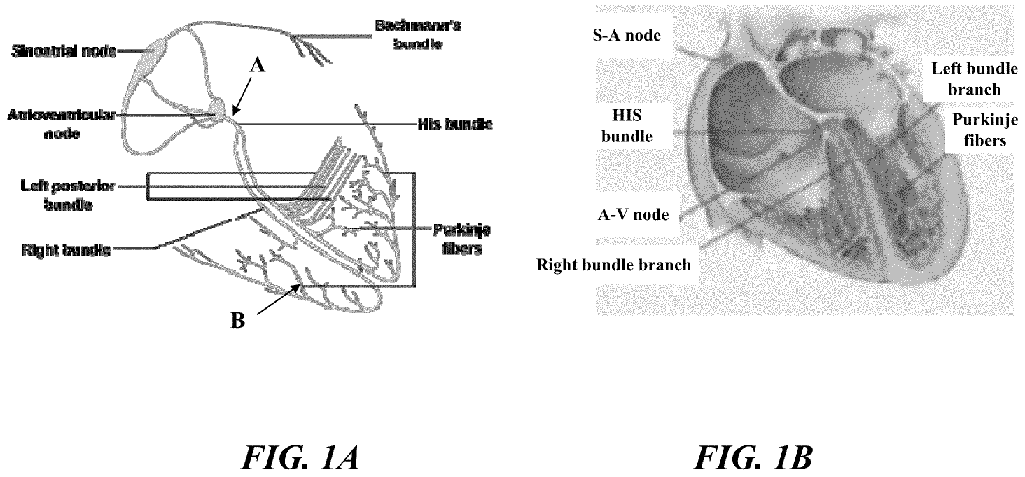

As illustrated in FIG. 1A, Purkinje fibers are specialized myocardial conduction fibers that arise from the bundle branches and spread the impulse to the myocardial contraction fibers of the ventricles. Although Purkinje fiber and HIS bundle cell types are similar, their geometry is different. HIS bundle pacing is injected at point A. The Purkinje system may be entered from the right ventricular apex at point B. Experimental results were conducted in humans and show that pacing through the Purkinje system is possible at lower voltages. FIG. 1B depicts a cross section of the heart showing the HIS bundle and the Purkinje fibers.

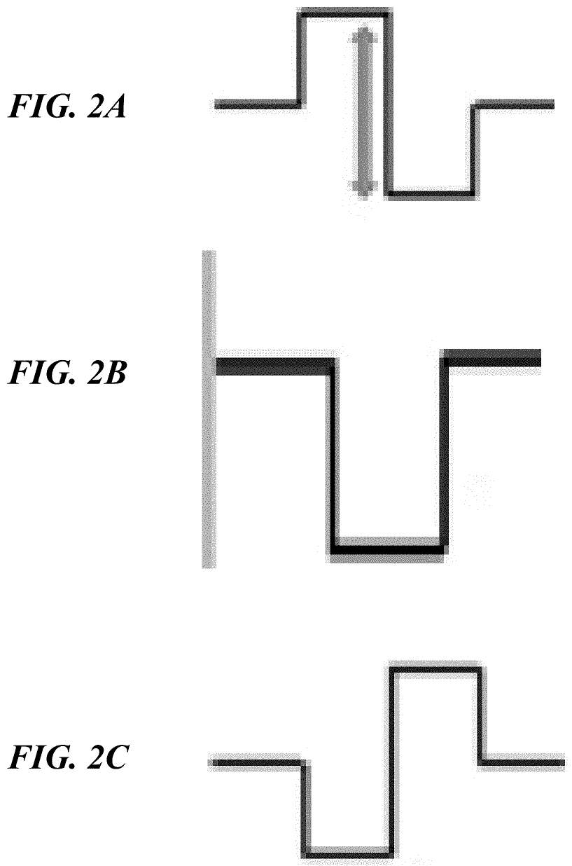

A biphasic anodal/cathodal pacing waveform is shown in FIG. 2A. FIG. 2B shows a cathodal only pacing waveform and FIG. 2C shows a biphasic cathodal/anodal waveform.

An experiment was conducted in humans in which the Purkinje fibers were stimulated with a biphasic anodal/cathodal pacing waveform. In patient 1, a 0.5 msec/0.5 msec anodal/cathodal waveform compared to a monophasic cathodal pulse showed increased speed of 72 msec compared to 83 msec (a 15% increase). Compared to thresholds of 2 volts to 3 volts, the threshold of a 0.1 msec/1.8 msec short/long waveform was 0.5 volts. In patient 2, compared to thresholds of 1-2 volts, the threshold for the 0.1 msec/1.8 msec short/long waveform was <0.5 volts, independent of polarity. No gross ECG changes were noted in either patient.

These results indicate that pacing in the Purkinje system is conducted with a fraction of the energy usually required. As pacemaker batteries have limited lifetime, pacing with lower voltage waveforms can extend battery life and reduce the number of operations a patient must have to maintain the pacemaker. The biphasic anodal/cathodal pacing of the Purkinje system decreased the battery drain by 80% as compared to the battery drain using cathodal only pacing.

These results are summarized in Table 1.

TABLE 1
Summary of Bi-Phasic Pacing in the Purkinje System

| | | Threshold | | | |
|---|---|---|---|---|---|
| Subject | Type | Voltage | Pulse Lengths | Speed | % |
| Patient 1 | Anodal/Cathodal | | 0.5 ms/0.5 ms | 72 ms | 15 |
| Patient 1 | Anodal/Cathodal | 0.5 volts | 0.1 ms/1.8 ms | | |
| Comparison 1 | Cathodal Only | 2-3 volts | Monophase 0.5 msec | 83 ms | |
| Patient 2 | Anodal/Cathodal | <0.5 volts | 0.1 ms/1.8 ms | | |
| Comparison 2 | Cathodal Only | 1-2 volts | Monophase 0.5 ms | 83 ms | |

In addition to confirming these effects in humans, waveforms with short pulse widths may preferentially enter the Purkinje conduction system because of its lower chronaxie than that of myocardial cells. The chronaxie is defined as the minimum amount of time needed to stimulate a muscle or nerve fiber, using an electric current twice the strength required to elicit a threshold response.

There are two major types of cardiac muscle cells: myocardial contractile cells and myocardial conducting cells. The myocardial contractile cells constitute the bulk (99 percent) of the cells in the atria and ventricles. Contractile cells conduct impulses and are responsible for contractions that pump blood through the body. The myocardial conducting cells (1 percent of the cells) form the conduction system of the heart. Except for Purkinje cells, they are generally much smaller than the contractile cells and have few of the myofibrils or filaments needed for contraction. Their function is similar in many respects to neurons, although they are specialized muscle cells. Myocardial conduction cells initiate and propagate the action potential (the electrical impulse) that travels throughout the heart and triggers the contractions that propel the blood. Adding an anodal component to pacing waveforms may enhance this conducting system entry effect.

Although the Purkinje fiber and the HIS bundle cell types are similar, their geometry is different. Referring to FIG. 1A, HIS bundle pacing is done from site A. In aspects of the present disclosure, the pacing waveforms enter at site B, where the number of fibers is much fewer than myocardial cells when compared to areas in which there is more of a cable arrangement (and there is some separation between the myocardial cells and the cable) like the HIS bundle (A) and the right bundle (B).

The low threshold voltage of the pacing waveforms of Table 1 indicates that conduction in the Purkinje tissue was activated, which directly stimulated the myocardium, simulating non-selective stimulation. The voltage threshold for stimulating the Purkinje system cells is lower than the voltage threshold for direct stimulation of the myocardial cells during pacing.

These results show that biphasic waveform pacing:
i. Reduces the adverse effects of standard cathodal pacing,
ii. Increases the speed of the conduction of the pacing stimuli,
iii. Increases the strength of conduction of the pacing stimuli,
iv. Increases cardiac output,
v. Reduces heart failure,
vi. Uses less energy,
vii. May treat atrial fibrillation (AF),
viii. Improves heart function,
ix. May have post myocardial infarction applications.

Retrograde conduction is a conduction backward phenomena in the heart, where the conduction comes from the ventricles or from the atrial valve (AV) node into and through the atria. In the lower voltage pacing of Table 1, it was found that not enough Purkinje fibers were excited to stimulate retrograde conduction. A screw-in electrode may be directly connected to the right bundle (site B) to increase the retrograde conduction.

Although various patterns of HIS bundle capture differ from each other, these may be extremely subtle and can easily be missed on cursory examination. Ancillary work in HIS bundle pacing shows several patterns of QRS patterns depending on the various mixtures of myocardial and HIS bundle captures.

The QRS complex is the combination of three of the graphical deflections seen on a typical electrocardiogram (ECG or EKG). It is usually the central and most visually obvious part of the tracing; in other words, it's the main spike seen on an ECG line. It corresponds to the depolarization of the right and left ventricles of the human heart and contraction of the large ventricular muscles.

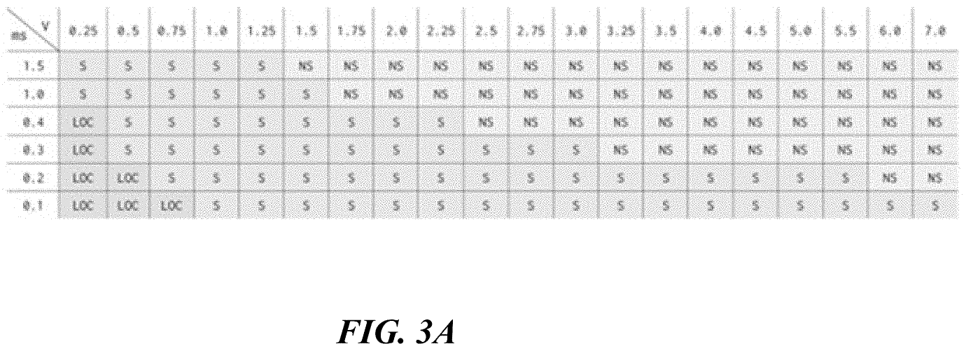

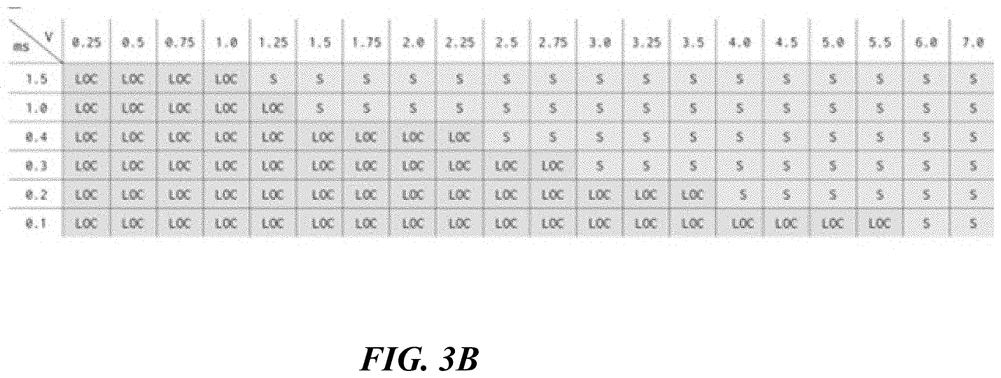

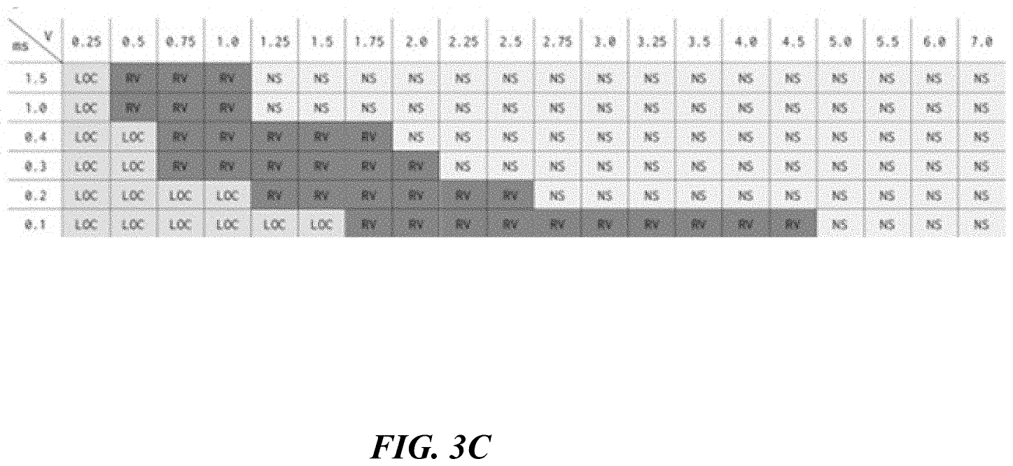

HIS bundle pacing has also been shown to benefit from biphasic anodal/cathodal pacing. FIGS. 3A-3C illustrate threshold assessment charts for patients with permanent HIS bundle (HB) pacing showing three distinct patterns of capture with regard to selective/nonselective capture. FIG. 3A represents a patient with a wide zone of selective pacing that broadens with a shorter pulse width. FIG. 3B represents a patient with obligatory selective capture. FIG. 3C represents a patient with obligatory nonselective HB capture. In FIG. 3A-3C, LOC represents “loss of capture”, S represents “selective HIS bundle pacing”, NS represents “non-selective HIS bundle pacing”, and RV represents “right ventricle only pacing”.

In selective HIS bundle pacing (S-HBP), an isoelectric interval is visible in all leads, that corresponds to the HIS-ventricular (HV) interval and separates the pacing spike from QRS onset. The QRS morphology is most often identical to that in intrinsic rhythm.

In nonselective HIS bundle pacing (NS-HBP), the lead is usually positioned in the ventricle at a site where the HB is surrounded by or at proximity to myocardial tissue.

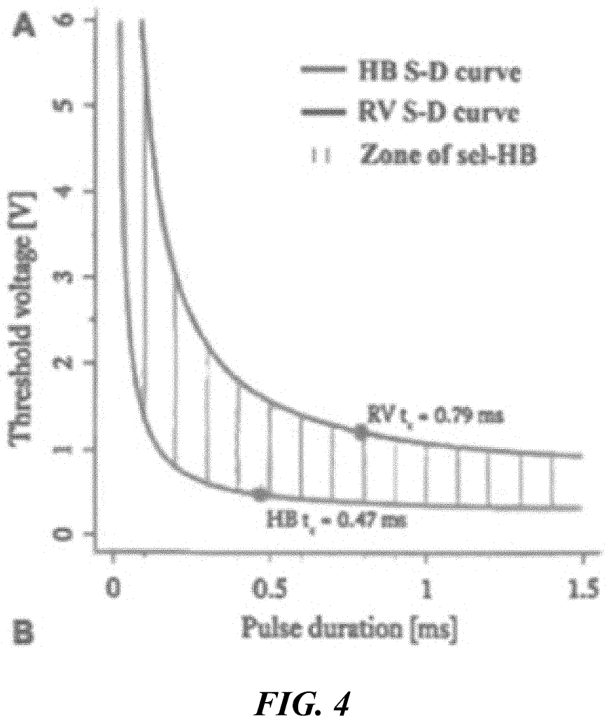

FIG. 4 is a graph comparing threshold voltage to pulse duration for HIS bundle pacing and right ventricle pacing. The zone between the curves represents selective HIS bundle pacing. In patients with selective HIS bundle (HB) pacing, the chronaxie for the HB is shorter than that for the right ventricular (RV) myocardium. Consequently, the zone of selective pacing widens with shortening of the pulse duration.

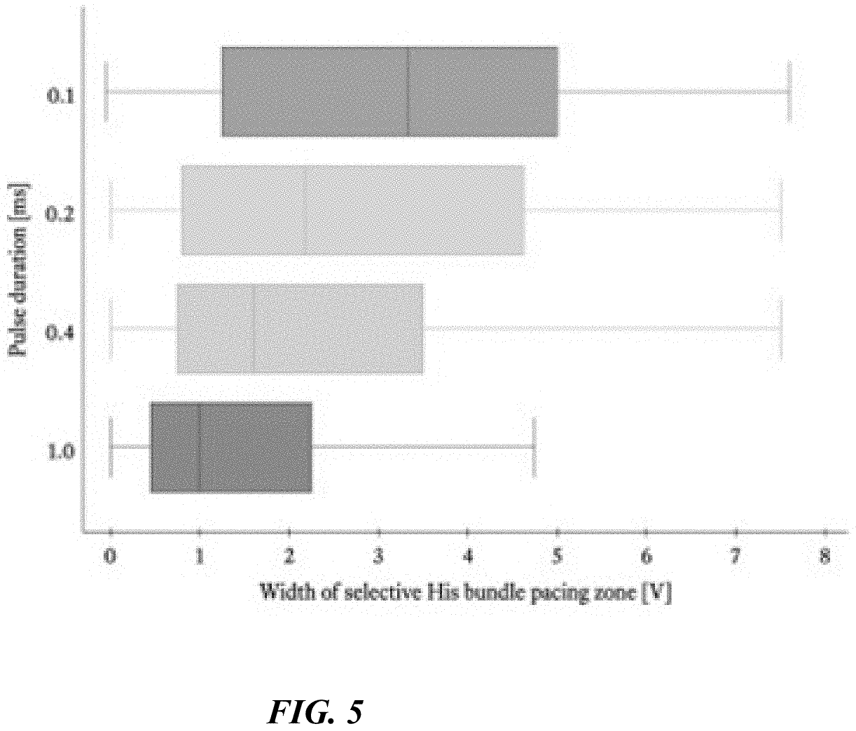

FIG. 5 compares pulse duration to width of the HIS bundle zone for selective HB pacing subgroup in a box plot with median and interquartile range from 25% to 75%. The zones of programmable selective HIS bundle capture are wider for shorter pulse durations. As high pacing thresholds are linked to faster battery depletion, pacing at a pulse duration closer to the chronaxie may decrease battery current duration and facilitate selective HB capture.

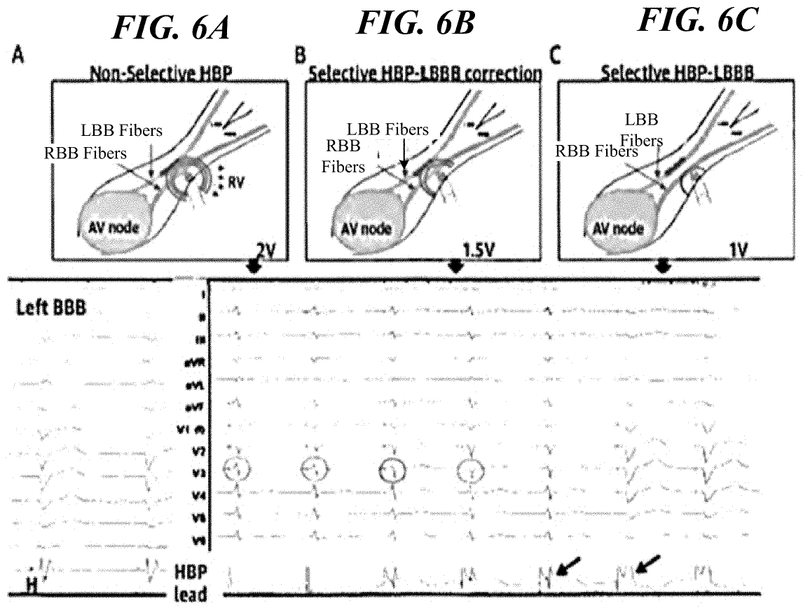

FIGS. 6A, 6B, 6C illustrate that fibers within the HIS bundle are already pre-destined to become the right bundle branch (RBB) and left bundle branch (LBB). (A) Pacing at 2V results in capture of local ventricular tissue and HIS (both RBB and LBB fibers), which is considered nonselective HBP. There is minimal delta wave on the surface electrocardiogram (ECG) (blue circles). However, ventricular capture is evidenced by the absence of local electrogram in the HIS bundle packing (HBP) lead. (B) Pacing at 1.5 V results in selective HIS (RBB and LBB) capture (no delta wave, as in orange circles) with loss of ventricular capture (arrow shows discrete local electrogram in the HBP lead). (C) Pacing at 1.0 V demonstrates capture of RBB fibers alone with LBB block pattern (arrow shows the discrete local electrogram with different morphology).

Cardiac resynchronization therapy (CRT) aims at three different levels (a) atrial valve (AV) level (b) intraventricular level and (c) the interventricular level. This is achieved by pacing or sensing the right atrium, pacing the right ventricle (near the interventricular septum) and pacing the left ventricle (using the coronary venous branches), also called biventricular pacing.

In a related area of research, bi-ventricular pacing (also called cardiac resynchronization therapy) has been used to stimulate pacing. In bi-ventricular pacing, leads are implanted through a vein into the right ventricle and into the coronary sinus vein to pace or regulate the left ventricle. Usually a lead is also implanted into the right atrium. Bi-ventricular pacing keeps the right and left ventricles pumping together by sending small electrical impulses through the leads. The above results show that bi-ventricular pacing may be used to improve myocardial dysfunction by forcing all the heart muscles to beat synchronously. Biventricular pacing for treatment of congestive heart failure was first applied clinically in 1991. The initial five implants used a bipolar Y-adapter, pacing one ventricle with the cathode and the other with the anode. Quite unexpectedly, the pacing thresholds gradually rose at the anode and, in 4-6 weeks, threshold exceeded the output of the generator, at which time pacing at the anode was lost. When this occurred, heart failure returned and the Y-adapter had to be replaced with a split-cathode design, thereby allowing pacing of both sites with the cathode. Upon completion of this procedure, pacing of both ventricles resumed and the heart failure again resolved. Patients implanted thereafter used only the split-cathode design. However, the resolution of the congestive heart failure seemed to occur more slowly than when there had been the presence of the anodal phase, suggesting that there might be some additional beneficial properties of having an anodal component. Experiments in a variety of animal species have indicated that anodal stimulation alone or as part of a biphasic anodal/cathodal waveform gives rise to driven beats that travel faster over the myocardium and enhance contractility.

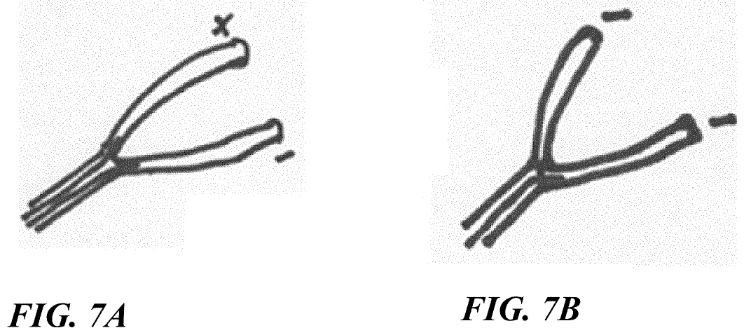

FIGS. 7A, 7B show two types of biventricular electrode arrangements, an extended bipole (FIG. 7A) and a split bipole (FIG. 7B). The extended bipole provides biphasic anodal/cathodal pacing and has been shown to provide faster resynchronization.

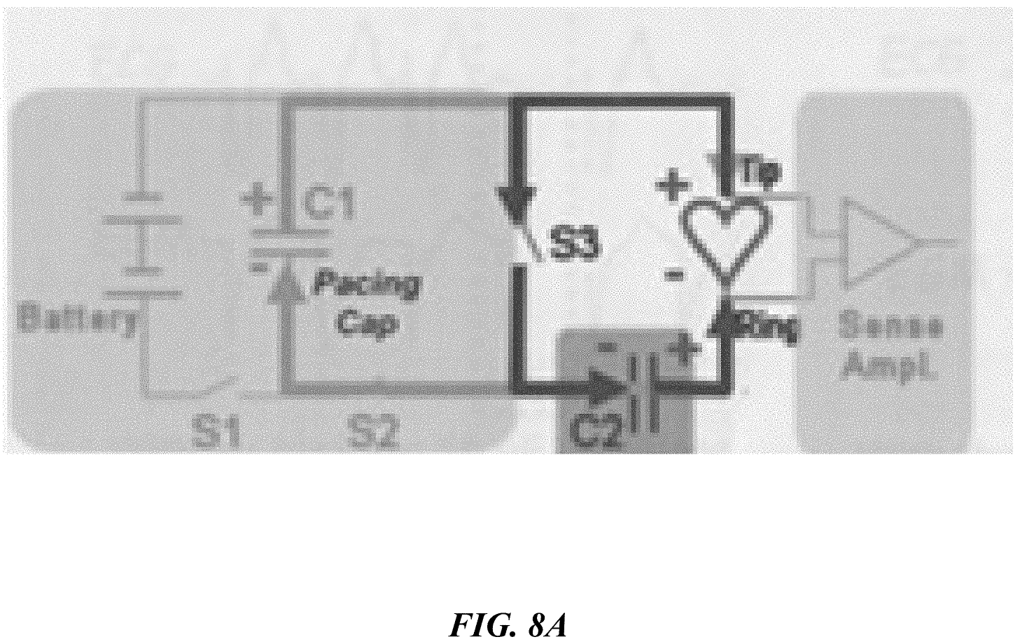

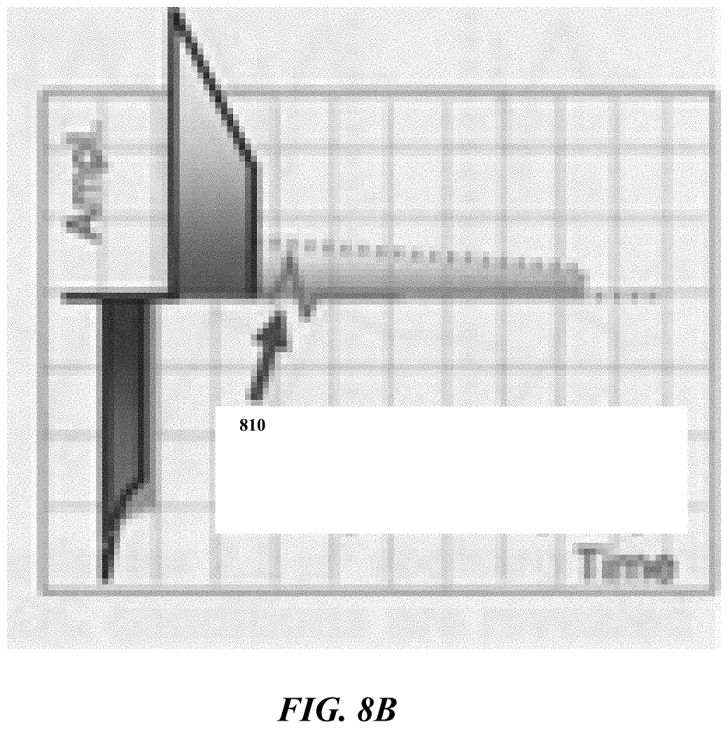

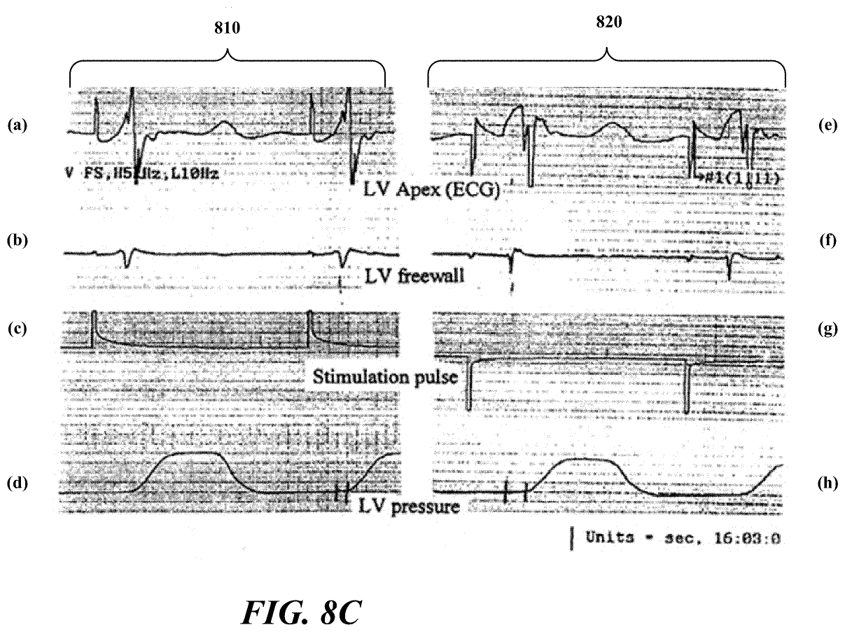

FIG. 8A shows a circuit diagram for biphasic pacing using a biventricular electrode for HIS bundle pacing. The positive electrode is referred to as the “Tip” and is placed at the atrioventricular node. The negative electrode is referred to as the “Ring” and is placed at the HIS bundle. Circuit elements include a battery, pacing capacitor C1, coupling capacitor C2 and sense amplifier having leads across the positive “Tip” and negative “Ring” electrodes.

FIG. 8B shows the amplitude with respect to time for a cathodal/anodal biphasic pulse and the evoked response 810 (area under the dotted lines).

FIG. 8C shows an electrocardiogram depicting (a) an anodal pulse 810, (b) the response to the anodal pulse at the left ventricle free wall, (c) the stimulation response pulse to the anodal pulse, (d) the left ventricle pressure resulting from the stimulation by the anodal pulse, (e) a cathodal pulse 820, (f) the response to the cathodal pulse at the left ventricle free wall, (g) the stimulation response pulse to the cathodal pulse, (h) the left ventricle pressure resulting from the stimulation by the cathodal pulse.

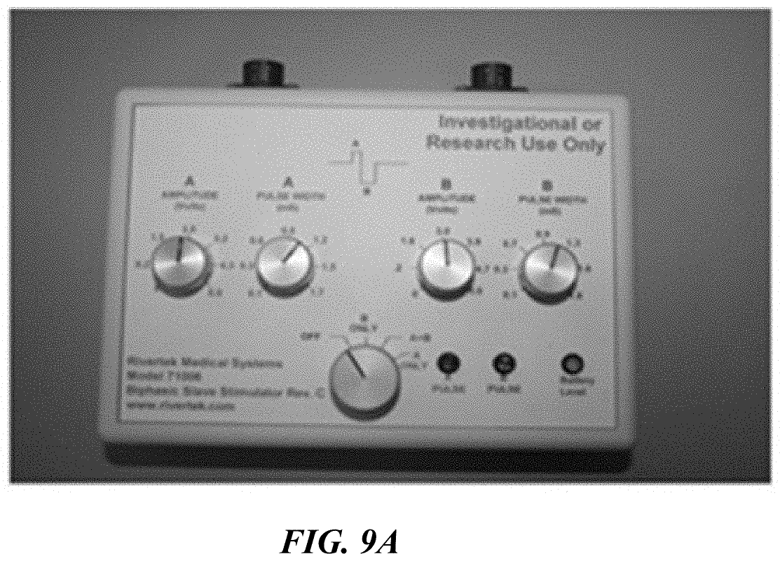

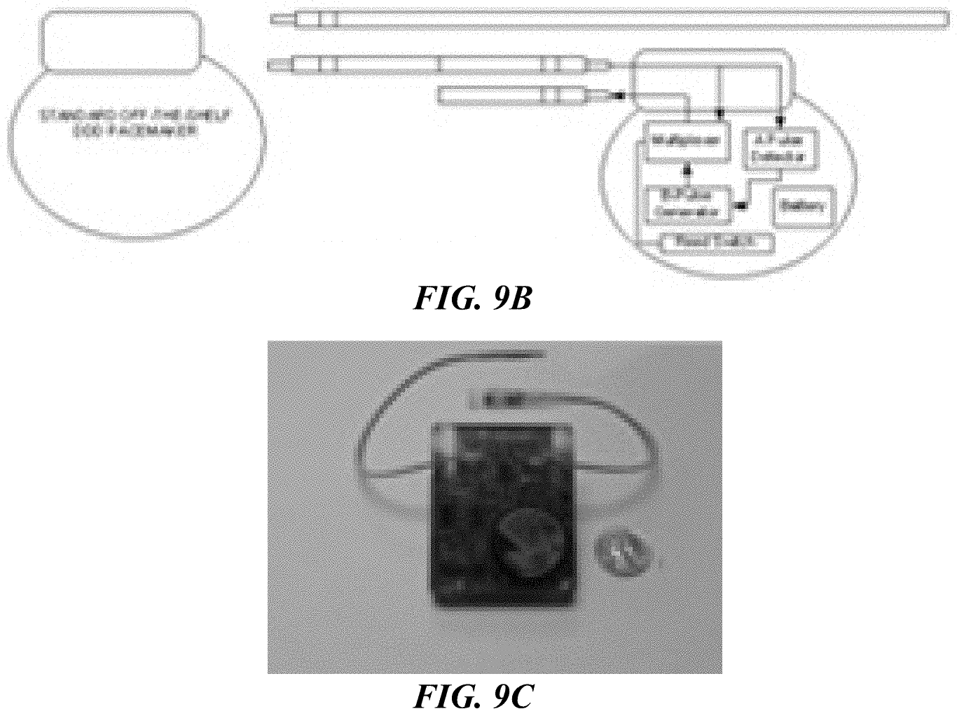

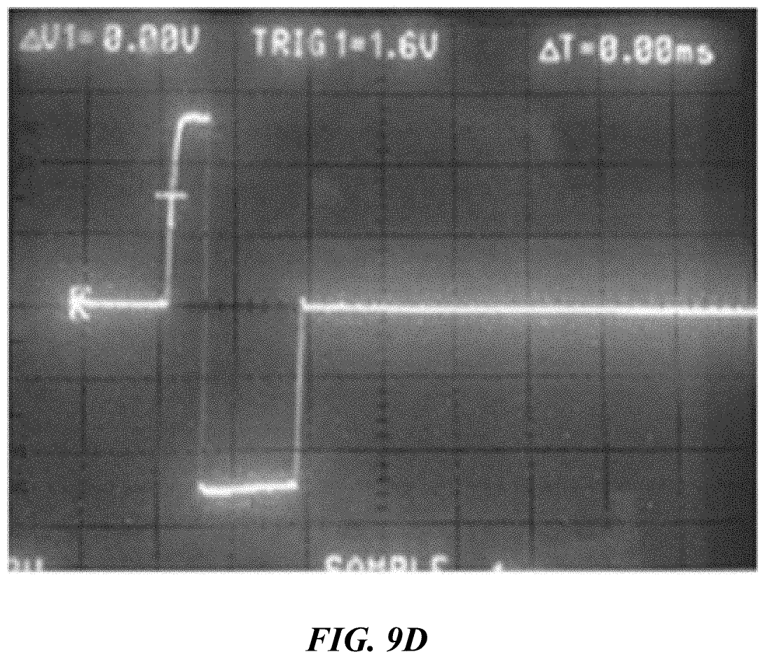

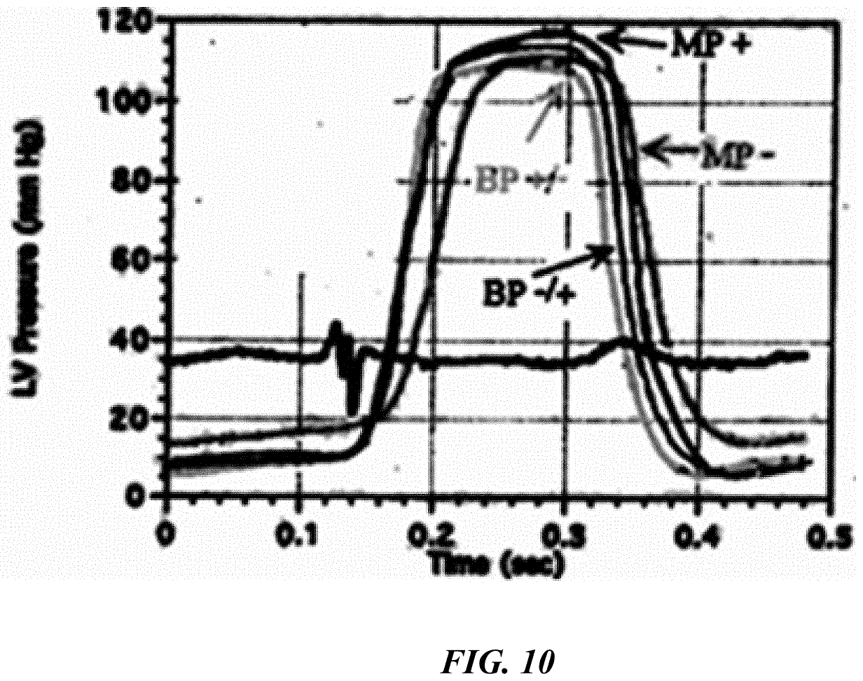

FIG. 9A shows an external waveform stimulator which was used to stimulate the biphasic pulses during experiments.

FIG. 9B illustrates an implantable waveform stimulator showing the leads and the internal pacemaker circuit.

FIG. 9C shows a pacemaker circuit board and the comparison in size to a U.S. quarter.

FIG. 9D shows an oscilloscope picture of a biphasic anodal/cathodal pulse of the pacemaker of FIG. 9C. FIG. 10 shows a graph of left ventricle pressure with respect to time of four response pulses, i.e., a biphasic cathodal/anodal pulse, a biphasic anodal/cathodal pulse, an negative membrane potential and a positive membrane potential. Since all cells are more electronegative in their interior as opposed to exterior, pacing which increases the membrane potential before actual depolarization gives better performance in terms of speed of conduction, contractility and intercellular functions.

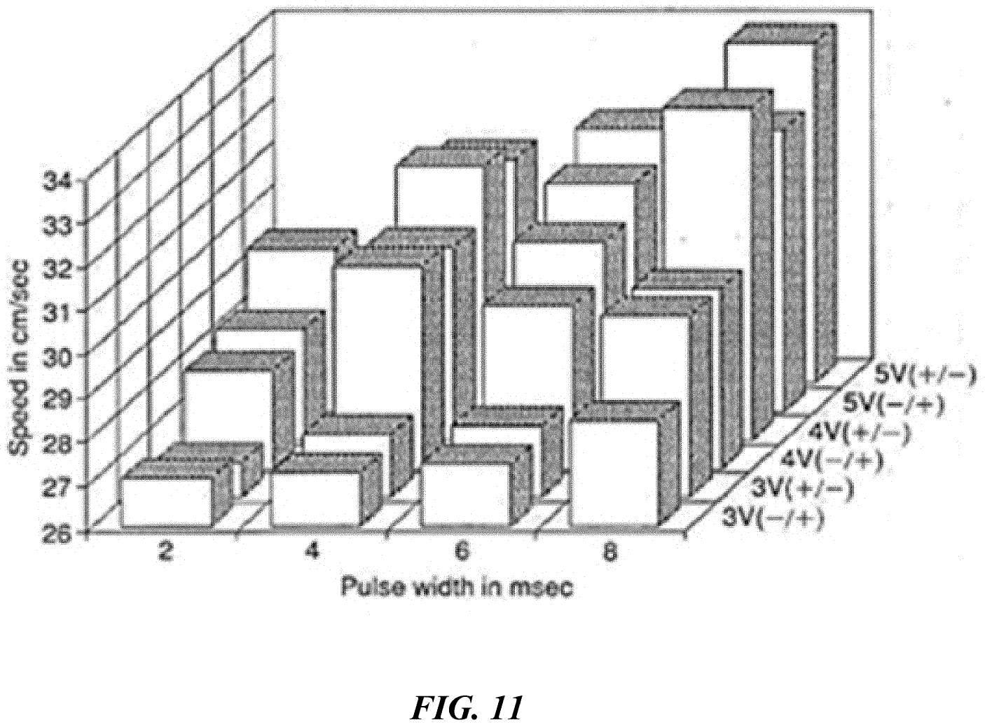

FIG. 11 shows experimental results of using biphasic stimulation in HIS bundle pacing. The speed of conduction (cm/sec) with respect to pulse width (msec) at a 3 volt cathodal/anodal pulse and a 3 volt anodal cathodal pulse, a 4 volt cathodal/anodal pulse and a 4 volt anodal cathodal pulse, and for a 5 volt cathodal/anodal pulse and a 5 volt anodal cathodal pulse. This graph shows that an increase in pulse width increased the speed of conduction, that an increase in voltage increased the speed of conduction and that the anodal/cathodal pulse significantly improved the speed of conduction over the cathodal/anodal pulse.

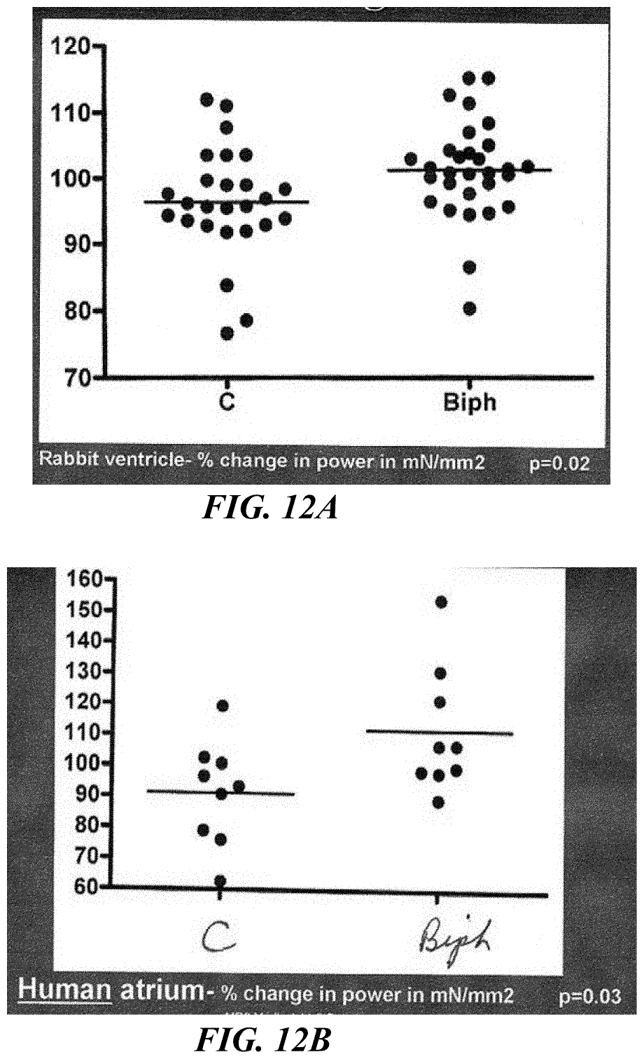

FIG. 12A shows the change in contractility in isolated muscle strips of a rabbit ventricle due to cathodal (left) and biphasic (right) pulses and FIG. 12B shows the change in power in isolated muscle strips of a human atrium due to cathodal (left) and biphasic pulses (right). In both figures, the biphasic pulse resulted in higher contractility of the muscle strip.

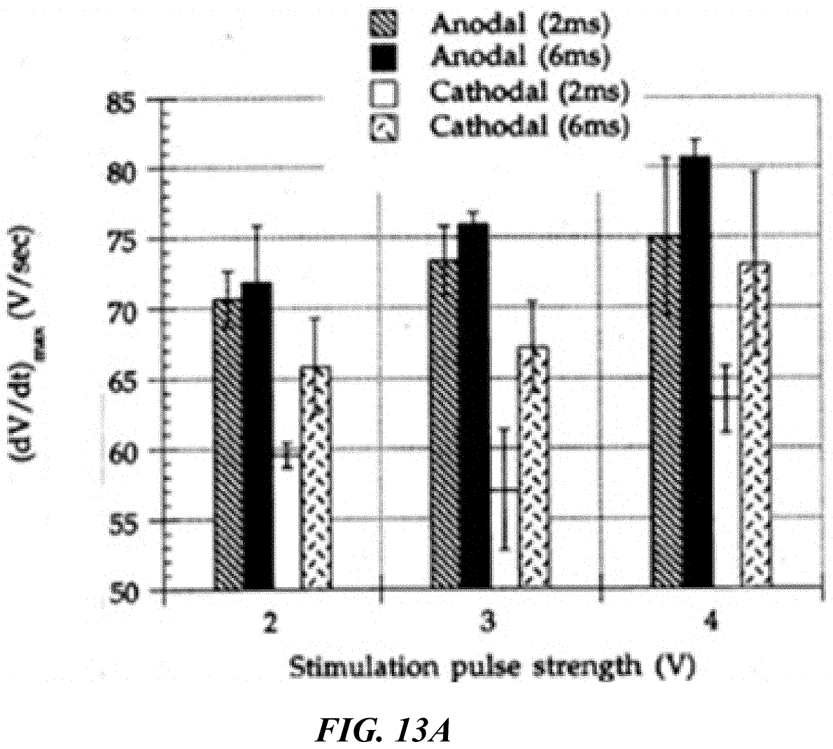

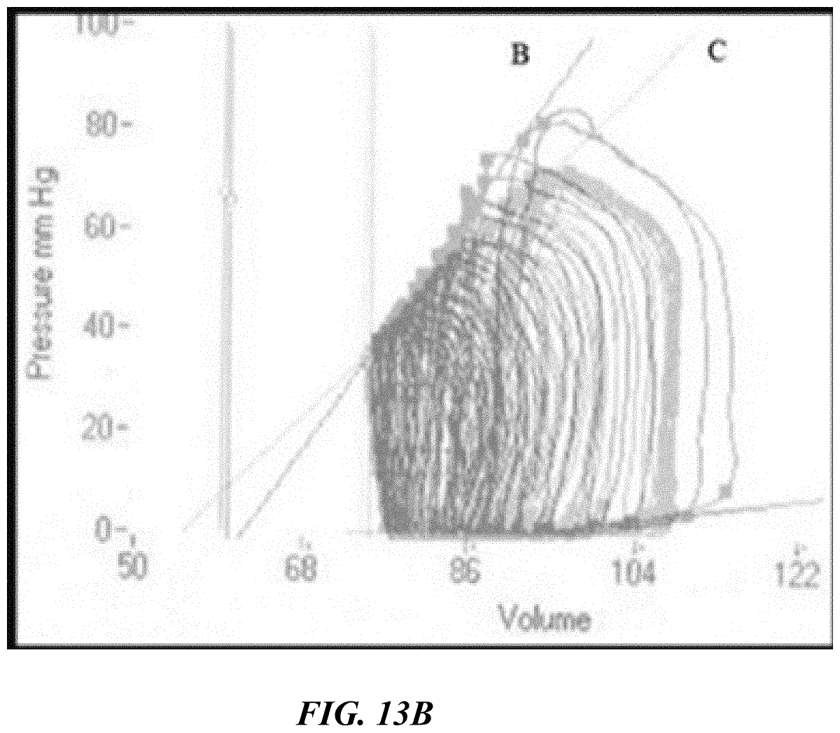

FIG. 13A is a graph from rabbit heart experiments showing the change in conduction velocity (V/sec) with respect to stimulation pulse strengths of 2, 3 and 4 volts. In each case, the anodal pulses at 2 ms and 6 ms showed much greater response than for the cathodal pulses.

FIG. 13B is a graph from rabbit experiments of cardiac pressure with respect to cardiac volume for biphasic (B) and cathodal only (C) waveforms. This graph demonstrates that the biphasic anodal/cathodal pulse generates greater cardiac contractility than the cathodal only pulse.

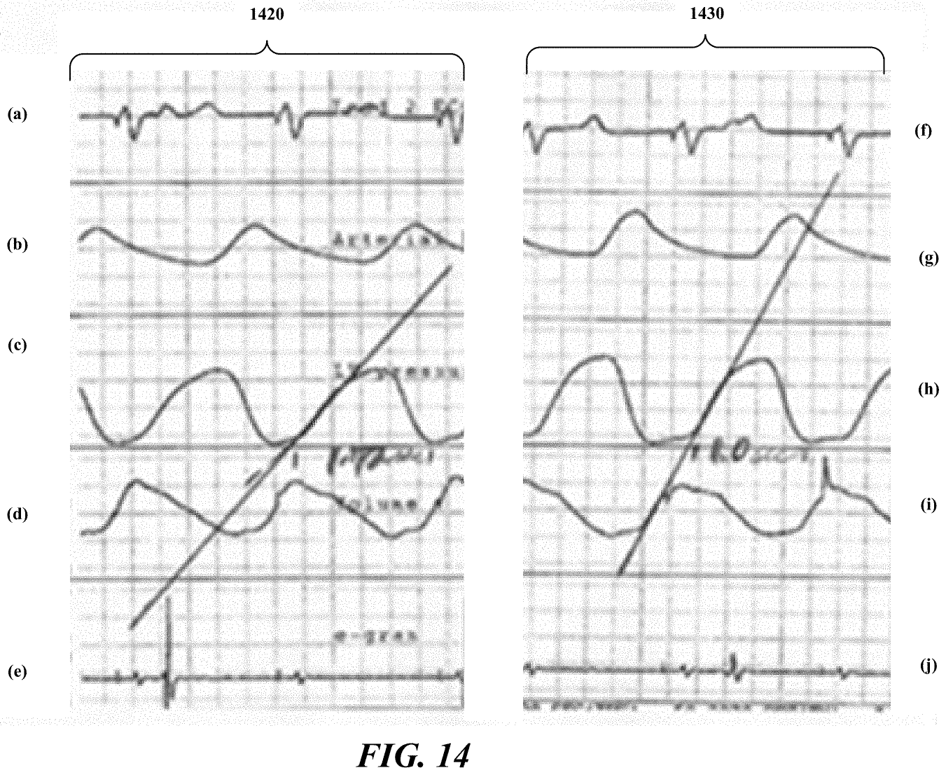

Research in cardiac pacing performed on pigs which compared the contractility and relaxation due to cathodic only and biphasic cathodal/anodal waveforms demonstrated that the biphasic waveform increased contractility and relaxation. Upstroke (dP/dt+) and relaxation (dP/dt2) of left ventricular pressure curve were measured in two 50 kg subjects paced slightly above intrinsic rate with cathodal (C) and anodal followed by cathodal biphasic (B) pacing pulses. FIG. 14 shows comparison of cardiac pacing in pig hearts with a cathodal pulse only 1420 to pacing with biphasic cathodal/anodal waveforms 1430. In FIG. 14, (a) and (f) depict the cathodal pulse and the biphasic pulse, respectively, (b) and (g) compare the stimulation responses, respectively, (c) and (h) compare the left ventricle pressures, respectively, (d) and (i) compare the volumes of upstroke and relaxation, for the cathodal and biphasic stimulations, respectively, and (e) and (g) show the respective electrocardiograms.

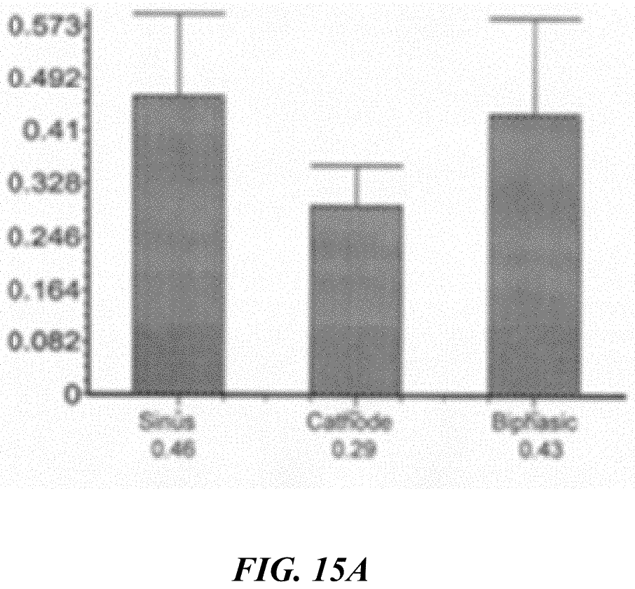

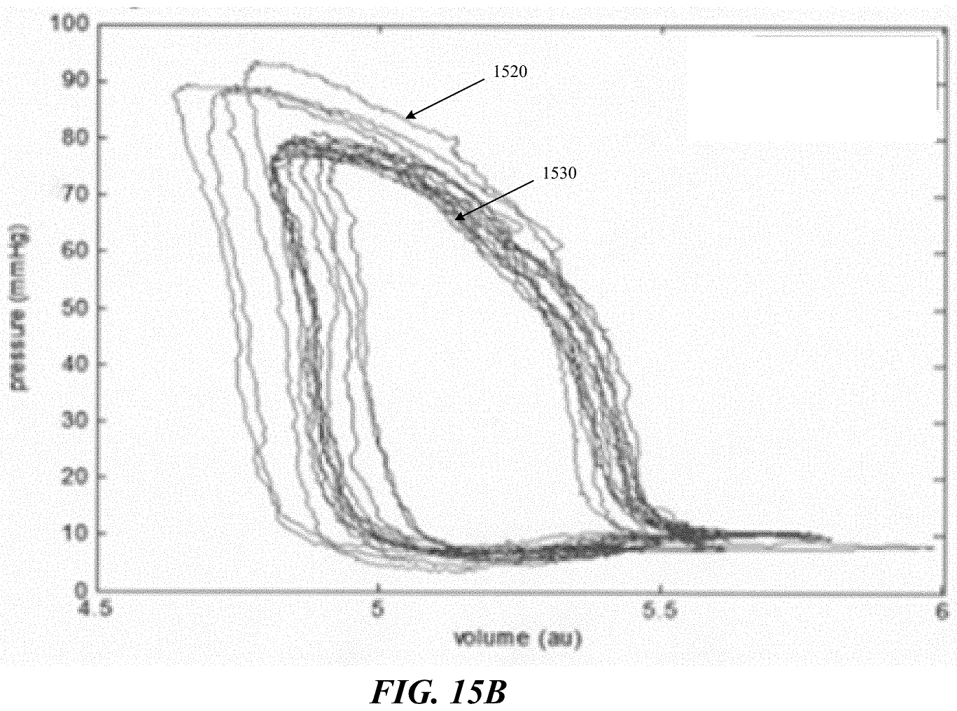

FIG. 15A is a box and whiskers graph comparing the mean and standard deviation of the steady state “preload-adjusted PWRmax” (maximal ventricular power divided by the square of end diastolic volume), which measures left ventricular contractility in a beat to beat fashion in atrial fibrillation. A corononary sinus lead was inserted in addition to a cathode lead and biphasic leads. A coronary sinus lead allows cardioversion and/or defibrillation stimuli to be provided by a large surface area electrode which is passively implanted in the coronary sinus, to allow the pulse generator to provide appropriately synchronized atrial-ventricular pacing, cardioversion or defibrillation. The SA (sinus) node represents a cluster of myocytes with pacemaker activity. Under normal circumstances, it generates electrical impulses that set the rhythm and rate of the heart. The main function of the SA node is to act as the normal pacemaker of the heart. It initiates an action potential that results in an electrical impulse traveling through the electrical conduction system of the heart to cause myocardial contraction. Unlike atrial and ventricular cells, pacemaker cells in the sinus node do not have a resting phase. Instead, these cells have pacemaker potential, in which they begin to depolarize automatically after an action potential ends.

FIG. 15A shows the results of experiments in pig hearts in which the sinus rhythm is compared to the pacing modalities. The sinus shows the largest PWRmax at 0.46, the biphasic pulse the second largest at 0.43 and the cathodal only pulse generated the smallest at 0.29. Table 2 compares the sinus, cathodal and biphasic measurement differences. In Table 2, Pmax refers to maximum pressure, SEP refers to systolic ejection pressure, EDP refers to end diastolic pressure, EDV refers to end diastolic volume and SW refers to stroke work.

TABLE 2
Comparison of Sinus, Cathodal and Biphasic Values

| | Pmax | SEP | EDP | EDV | SW |
|---|---|---|---|---|---|
| Sinus | 87.3 | 646 | 8.07 | 118 | 1650 |
| Cathodal | 84.9 | 551 | 8.3 | 118.9 | 1672 |
| Biphasic | 75.6 | 471 | 4.76 | 109.3 | 1034 |

FIG. 15B depicts pressure and volume loops in comparative stroke experiments using cathodal and biphasic pulses. To generate a PV loop for the left ventricle, the LV pressure is plotted against LV volume at multiple time points during a single cardiac cycle. The biphasic curves show smaller pressure per unit volume, which indicates lower cardiac work.

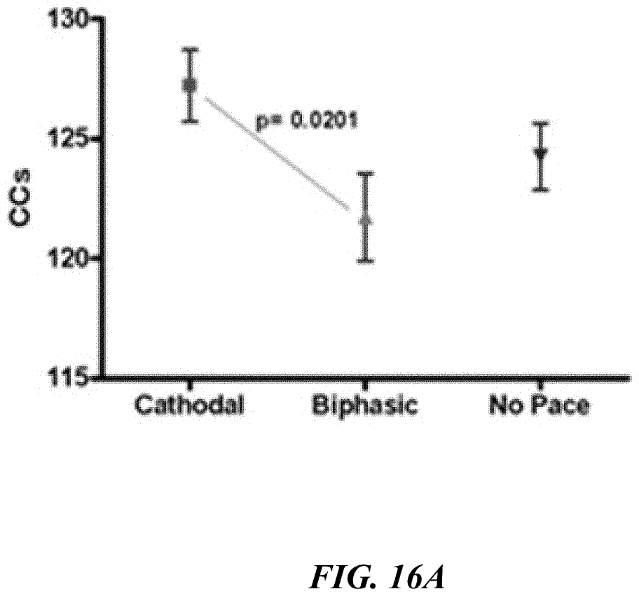

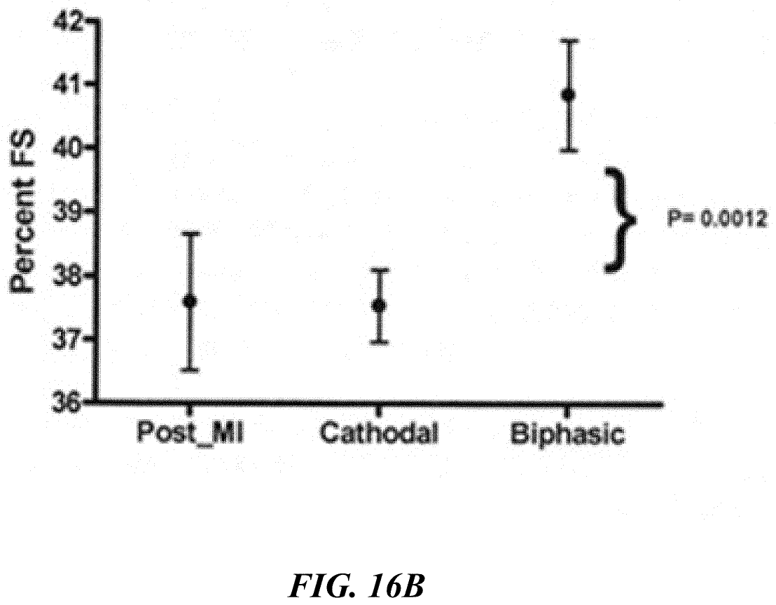

In experiments with sheep, biphasic pulsing elicited significant benefits in cardiac performance. FIG. 16A shows a comparison of cathodal, biphasic and unpaced left ventricle diastolic volumes, in which the biphasic diastolic volume was significantly lower than the cathodal and unpaced volumes. These results indicate that the heart had to do less work to pump the blood. The mean difference between the volumes for cathodal pacing and biphasic pacing was 5.5 cc. The cathodal pacing was conducted over 12.6 weeks, the biphasic over 10.9 weeks and the unpaced results were collected over 18 weeks.

FIG. 16B shows the changes in percent fractional shortening at nine weeks for the experiments of FIG. 16A. Fractional shortening is a percentage comparing the size differences in the left ventricle as a parameter of how well the left ventricle is contracting and reducing in size during systole. The fractional shortening is significantly higher for the biphasic pacing (mean 41%) compared to post myocardial infarction (post_MI) and cathodal pacing at about 37.5 percent.

Anodal burst pacing refers to a large initial pulse at an anodal electrode when treating atrial fibrillation. Experiments using anodal burst pacing in dogs showed 20% reversion of acetylcholine induced atrial fibrillation.

Experiments on human patients using biphasic burst pacing showed a 10% reversion of chronic atrial fibrillation.

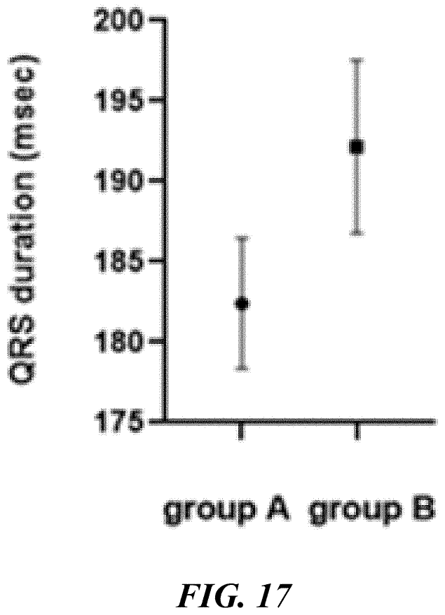

FIG. 17 shows an experiment on humans of using biphasic anodal/cathodal cardiac pacing in the atrium and in the ventricle for narrowing the paced QRS duration. The biphasic pacing narrowed the QRS duration by an average of 10.2 msec.

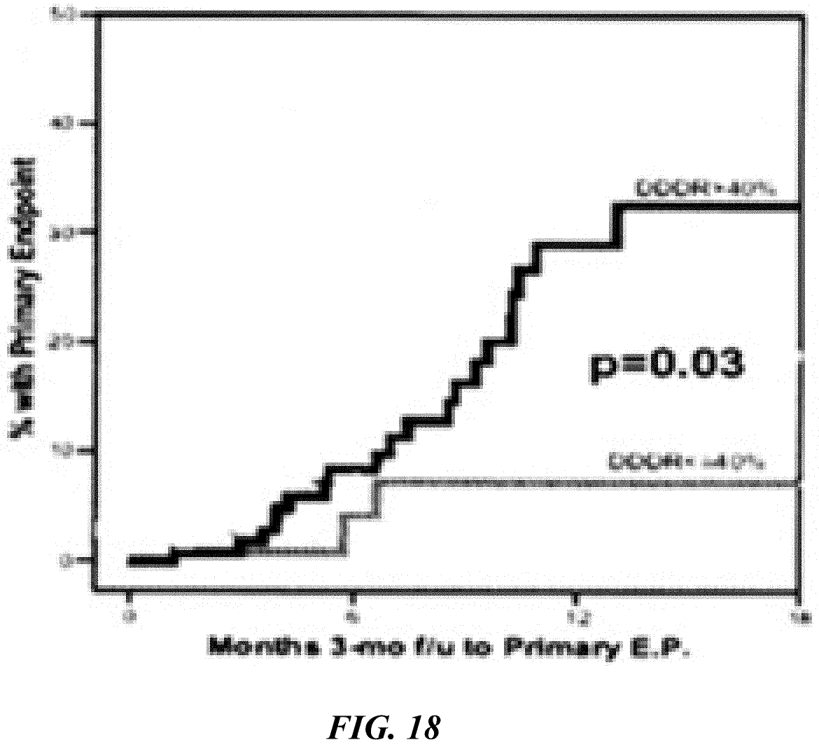

FIG. 18 shows a graph of mortality rates versus months from admission for patients having dual chamber sensing and stimulating greater than 40% of the time.

Experiments of cardiac pacing in humans for patients having ablation for atrial fibrillation were conducted. Biphasic anodal/cathodal pacing demonstrated left ventricle pacing with only 20% of the battery drain as compared to the battery drain during cathodal only pacing.

Additionally, the negative effects of cathodal only pacing are well recognized. Cathodal pacing causes inflammation and reduces cardiac contractility over time.

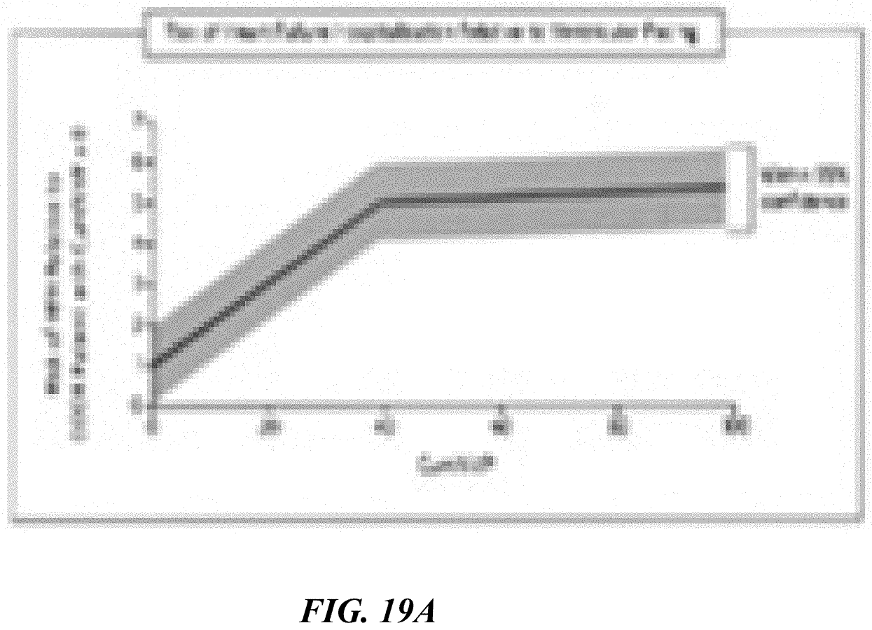

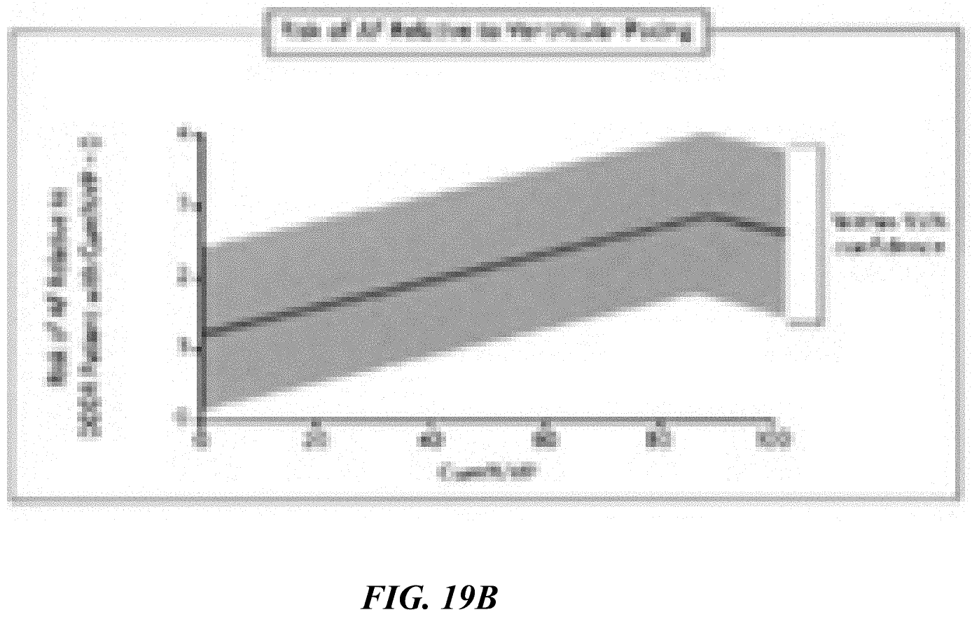

VVI pacing is ventricular demand pacing. The ventricle is paced, sensed, and the pulse generator inhibits pacing output in response to a sensed ventricular event. This mode of pacing prevents ventricular bradycardia and is primarily indicated in patients with atrial fibrillation with a slow ventricular response. Experiments have shown that with VVI pacing in ICDs (implantable defibrillators) (in patients without congestive heart failure (CHF)), those paced at greater than 50% had significant (20%) increased incidence of CHF compared to 9% in those with less pacing. FIG. 19A shows the increased risk of CHF for patients without congestive heart failure. FIG. 19B shows the increased risk of atrial fibrillation for patients without congestive heart failure.

Further, experiments have shown that there are negative effects of DDDR (rate responsive dual chamber sensing and stimulating) greater that 40% of the time. FIG. 18 shows the mortality rate over time. Further, research has shown that bipolar CRT pacing reduces adverse outcomes in the left bundle branch block.

An embodiment of the present disclosure describes biphasic anodal/cathodal pacing in human hearts wherein an electrode is placed in the right bundle to stimulate Purkinje fibers.

An embodiment of the present disclosure describes cardiac pacing via the distal Purkinje system with ultra-short pulse widths using a fraction of the energy needed for capture enabling much longer battery life.

A further embodiment of the present disclosure describes a screw-in electrode directly connected to the right bundle (site B) in human hearts to increase the retrograde conduction in anodal electrode biphasic pacing of the heart when stimulating Purkinje fibers.

An embodiment of the present disclosure describes HIS bundle pacing in human hearts by biphasic anodal/cathodal pacing.

An embodiment of the present disclosure describes HIS bundle pacing of the mid-septum right bundle branch to enable retrograde conduction back through the AV node and down the left bundle thus enabling cardiac resynchronization from the right ventricle of a human heart.

An embodiment of the present disclosure describes improving pacing stimulation by applying a biphasic waveform with anodal-first component to speed conduction of pacing stimuli through the conduction system of the human heart.

An embodiment of the present disclosure describes increasing pacemaker battery lifetime by using biphasic anodal/cathodal pacing.

Embodiments of the present disclosure may also be as set forth in the following parentheticals.

(1) A method of cardiac pacing of a human heart, comprising: stimulating Purkinje fibers of the human heart by a biphasic waveform including an anodal pulse followed by a cathodal pulse.

(2) The method of (1), further comprising: applying the anodal pulse to an anodal electrode and applying the cathodal pulse to a cathodal electrode, wherein the anodal electrode and the cathodal electrode are located in a mid-septum of the heart.

(3) The method of (1) or (2), applying the anodal pulse for a pulse length of t milliseconds; and applying the cathodal pulse for a pulse length of k milliseconds, wherein t is greater than k, t is equal to k, or t is less than k.

(4) The method of any one of (1) to (3), wherein t is in the range of 0.1 milliseconds to 2.0 milliseconds and k is in the range of 0.1 millisecond to 2.0 milliseconds, preferably wherein t is in the range of 0.1 milliseconds to 0.5 milliseconds and k is in the range of 0.5 milliseconds to 1.8 milliseconds.

(5) The method of any one of (1) to (3), further comprising: sensing, by a pacemaker connected to the anodal and cathodal electrodes, electrical signals in the heart which indicate an onset of atrial fibrillation; and applying the biphasic anodal and the cathodal pulses when the pacemaker senses the onset of atrial fibrillation.

(6) The method of any one of (1) to (2), comprising: applying a square wave voltage of positive amplitude V₁ to the anodal electrode; and applying a square wave voltage of negative amplitude V₂ to the cathodal electrode, wherein V₁ is less than or equal to 0.05 V₂.

(7) The method of any one of (1) to (2), further comprising: stimulating retrograde conduction by applying the anodal pulse to a screw-in electrode located in the vicinity of the right bundle.

(8) A method of cardiac pacing of a human heart, comprising: stimulating the HIS bundle of the human heart by a biphasic waveform including an anodal pulse followed by a cathodal pulse.

(9) The method of (8), further comprising: stimulating the HIS bundle at the mid-septum right bundle branch to generate retrograde conduction through an atrioventricular (AV) node and down a left bundle, to resynchronize cardiac conduction from a right ventricle.

(10) The method of any one of (8) to (9), further comprising: stimulating the HIS bundle by an anodal pulse applied to a biventricular tip electrode located at an atrioventricular node; and stimulating the HIS bundle by a cathodal pulse applied to a biventricular ring electrode located around the HIS bundle.

(11) The method of any one of (8) to (10), comprising: applying a square wave voltage of positive amplitude V₁ to the anodal electrode; and applying a square wave voltage of negative amplitude V₂ to the cathodal electrode, wherein V₁ is less than or equal to V₂, and wherein the amplitudes of V₁ and V₂ are both within in the range of 1 volt to 5 volts.

(12) The method of any one of (8) to (11), further comprising: sensing, by a pacemaker connected to the anodal and cathodal electrodes, electrical signals in the heart which indicate an onset of atrial fibrillation; and applying the anodal and the cathodal pulses when the pacemaker senses the onset of atrial fibrillation.

(13) A method extending the lifetime of a pacemaker battery, comprising: generating, by a pulse generator of the pacemaker, a series of low voltage square wave biphasic waveforms each including an anodal pulse followed by a cathodal pulse, the biphasic waveforms having ultra short pulse widths; applying the anodal pulse to an anodal electrode located in a human heart, wherein the anodal pulse has a positive amplitude V₁; applying the cathodal pulse to an cathodal electrode located in a human heart, wherein the cathodal pulse has a negative amplitude V₂, wherein V₁ and V₂ are lower in amplitude than an amplitude of a voltage V₃ applied to the cathodic electrode by a single phase pacing waveform; and stimulating the human heart by the series of biphasic waveforms.

(14) The method of (13), further comprising: installing the positive and the negative electrodes in a mid-septum of the human heart; stimulating the Purkinje fibers of the human heart by applying the series of biphasic waveforms, wherein V₁ is less than or equal to 0.05 V₂.

(15) The method of any one of (13) and (14), further comprising: stimulating retrograde conduction in the Purkinje fibers by applying the anodal pulse to a screw-in electrode located in the vicinity of the right bundle.

(16) The method of any one of (13) to (15), further comprising: applying the square wave voltage of positive amplitude V₁ to a biventricular tip electrode located at an atrioventricular node; applying the square wave voltage of negative amplitude V₂ to a biventricular ring electrode located around a HIS bundle of the human heart, wherein V₁ is less than or equal to V₂, and wherein the amplitudes of V₁ and V₂ are each within in the range of 1 volt to 5 volts; and resynchronizing cardiac conduction by stimulating the HIS bundle at the mid-septum right bundle branch to generate retrograde conduction through the atrioventricular (AV) node and down a left bundle.

(17) The method of (13), further comprising: applying the anodal pulse for a pulse length of t milliseconds; and applying the cathodal pulse for a pulse length of k milliseconds, wherein t is greater than k, t is equal to k, or t is less than k.

(18) The method of any one of (13) and (17), wherein t is in the range of 0.1 milliseconds to 2.0 milliseconds and k is in the range of 0.1 millisecond to 2.0 milliseconds, preferably wherein t is in the range of 0.1 milliseconds to 0.5 milliseconds and k is in the range of 0.5 milliseconds to 1.8 milliseconds.

(19) The method of (13) further comprising: sensing, by a pacemaker connected to the anodal and cathodal electrodes, electrical signals in the heart which indicate an onset of atrial fibrillation; and applying the anodal and the cathodal pulses when the pacemaker senses the onset of atrial fibrillation.

(20) A method for cardiac resynchronization in a human heart, comprising: installing a large surface area electrode at a sinus node of the human heart; installing an anodal biventricular tip electrode at an atrioventricular node of the human heart; installing a cathodal biventricular ring electrode around a HIS bundle of the human heart; sensing, by a pacemaker connected to the anodal and cathodal electrodes, electrical signals which indicate an onset of atrial fibrillation; applying a positive pulse of voltage amplitude V₁ to the large surface area electrode; applying a pulse of positive amplitude V₂ to the anodal electrode followed by a negative pulse of amplitude V₃ to the cathodal electrode, wherein the amplitude of V₁ is greater than the amplitudes of V₂ and V₃, and wherein the amplitude of V₂ is less than or equal to the amplitude of V₃.

Obviously, numerous modifications and variations of the present invention are possible in light of the above teachings. It is therefore to be understood that within the scope of the appended claims, the invention may be practiced otherwise than as specifically described herein.

1. A method of cardiac pacing of a human heart comprising:
stimulating Purkinje fibers of the human heart by a biphasic waveform including an anodal pulse followed by a cathodal pulse.

2. The method of claim 1, further comprising:
applying the anodal pulse to an anodal electrode and applying the cathodal pulse to a cathodal electrode, wherein the anodal electrode and the cathodal electrode are located in a mid-septum of the heart.

3. The method of claim 2, further comprising:
applying the anodal pulse for a pulse length of t milliseconds; and
applying the cathodal pulse for a pulse length of k milliseconds, wherein t is greater than k, t is equal to k, or t is less than k.

4. The method of claim 3, wherein t is in the range of 0.1 milliseconds to 2.0 milliseconds and k is in the range of 0.1 millisecond to 2.0 milliseconds, preferably wherein t is in the range of 0.1 milliseconds to 0.5 milliseconds and k is in the range of 0.5 milliseconds to 1.8 milliseconds.

5. The method of claim 3, further comprising:
sensing, by a pacemaker connected to the anodal and cathodal electrodes, electrical signals in the heart which indicate an onset of atrial fibrillation; and
applying the biphasic anodal and the cathodal pulses when the pacemaker senses the onset of atrial fibrillation.

6. The method of claim 2, comprising:
applying a square wave voltage of positive amplitude V₁ to the anodal electrode; and
applying a square wave voltage of negative amplitude V₂ to the cathodal electrode, wherein V₁ is less than or equal to 0.05 V₂.

7. The method of claim 2, further comprising:
stimulating retrograde conduction by applying the anodal pulse to a screw-in electrode located in the vicinity of the right bundle.

8. A method of cardiac pacing of a human heart, comprising:
stimulating the HIS bundle of the human heart by a biphasic waveform including an anodal pulse followed by a cathodal pulse.

9. The method of claim 8, further comprising:
stimulating the HIS bundle at the mid-septum right bundle branch to generate retrograde conduction through an atrioventricular (AV) node and down a left bundle, to resynchronize cardiac conduction from a right ventricle.

10. The method of claim 9, further comprising:
stimulating the HIS bundle by an anodal pulse applied to a biventricular tip electrode located at an atrioventricular node; and
stimulating the HIS bundle by a cathodal pulse applied to a biventricular ring electrode located around the HIS bundle.

11. The method of claim 10, comprising:
applying a square wave voltage of positive amplitude V₁ to the anodal electrode; and
applying a square wave voltage of negative amplitude V₂ to the cathodal electrode, wherein V₁ is less than or equal to V₂, and wherein the amplitudes of V₁ and V₂ are both within in the range of 1 volt to 5 volts.

12. The method of claim 8, further comprising:
sensing, by a pacemaker connected to the anodal and cathodal electrodes, electrical signals in the heart which indicate an onset of atrial fibrillation; and
applying the anodal and the cathodal pulses when the pacemaker senses the onset of atrial fibrillation.

13. A method of extending the lifetime of a pacemaker battery, comprising:
generating, by a pulse generator of the pacemaker, a series of low voltage square wave biphasic waveforms each including an anodal pulse followed by a cathodal pulse, the biphasic waveforms having ultra short pulse widths;
applying the anodal pulse to an anodal electrode located in a human heart, wherein the anodal pulse has a positive amplitude V₁;
applying the cathodal pulse to an cathodal electrode located in a human heart, wherein the cathodal pulse has a negative amplitude V₂, wherein V₁ and V₂ are lower in amplitude than an amplitude of a voltage V₃ applied to the cathodic electrode by a single phase pacing waveform; and
stimulating the human heart by the series of biphasic waveforms.

14. The method of claim 13, further comprising;
installing the positive and the negative electrodes in a mid-septum of the human heart; and
stimulating the Purkinje fibers of the human heart by applying the series of biphasic waveforms, wherein V₁ is less than or equal to 0.05 V₂.

15. The method of claim 14, further comprising;
stimulating retrograde conduction in the Purkinje fibers by applying the anodal pulse to a screw-in electrode located in the vicinity of the right bundle.

16. The method of claim 13, further comprising:
applying the square wave voltage of positive amplitude V₁ to a biventricular tip electrode located at an atrioventricular node;
applying the square wave voltage of negative amplitude V₂ to a biventricular ring electrode located around a HIS bundle of the human heart, wherein V₁ is less than or equal to V₂, and wherein the amplitudes of V₁ and V₂ are each within in the range of 1 volt to 5 volts; and
resynchronizing cardiac conduction by stimulating the HIS bundle at the mid-septum right bundle branch to generate retrograde conduction through the atrioventricular (AV) node and down a left bundle.

17. The method of claim 13, further comprising:
applying the anodal pulse for a pulse length of t milliseconds; and
applying the cathodal pulse for a pulse length of k milliseconds, wherein t is greater than k, t is equal to k, or t is less than k.

18. The method of claim 17, wherein t is in the range of 0.1 milliseconds to 2.0 milliseconds and k is in the range of 0.1 millisecond to 2.0 milliseconds, preferably wherein t is in the range of 0.1 milliseconds to 0.5 milliseconds and k is in the range of 0.5 milliseconds to 1.8 milliseconds.

19. The method of claim 13, further comprising:
sensing, by a pacemaker connected to the anodal and cathodal electrodes, electrical signals in the heart which indicate an onset of atrial fibrillation; and
applying the anodal and the cathodal pulses when the pacemaker senses the onset of atrial fibrillation.

20. A method for cardiac resynchronization in a human heart, comprising:
installing a large surface area electrode at a sinus node of the human heart;
installing an anodal biventricular tip electrode at an atrioventricular node of the human heart;
installing a cathodal biventricular ring electrode around a HIS bundle of the human heart;
sensing, by a pacemaker connected to the anodal and cathodal electrodes, electrical signals which indicate an onset of atrial fibrillation;
applying a positive pulse of voltage amplitude V₁ to the large surface area electrode;
applying a pulse of positive amplitude V₂ to the anodal electrode followed by a negative pulse of amplitude V₃ to the cathodal electrode, wherein the amplitude of V₁ is greater than the amplitudes of V₂ and V₃, and wherein the amplitude of V₂ is less than or equal to the amplitude of V₃.
